(19) United States
(12) Patent Application Publication
(10) Pub. No.: US 2008/0125672 A1
(43) Pub. Date: May 29, 2008

(54) Low-Cost Hearing Testing System and Method of Collecting User Information

(76) Inventors: Mark Burrows, Princeton, NJ (US); John Cronin, Jericho, VT (US); Nancy Edwards, Jericho, VT (US); Steven D. Gabel, Sloatsburg, NY (US); Tushar Narsana, Winooski, VT (US); Steven A. Shaya, Highlands, NJ (US); John Anthony Singarayar, Skillman, NJ (US)

Correspondence Address: NORRIS MCLAUGHLIN & MARCUS, P.A., P O BOX 1018, SOMERVILLE, NJ, 08876, US

(21) Appl. No.: 11/570,466

(22) PCT Filed: Jun. 10, 2005
(86) PCT No.: PCT/US05/20437

§ 371 Date: Oct. 31, 2007

Related U.S. Application Data
(60) Provisional application No. 60/579,369, filed on Jun. 14, 2004.

(51) Int. Cl. A61B 5/12

(52) U.S. Cl. 600/559

(57) ABSTRACT

System and method for diagnosing hearing loss in an individual using a self-executable, interactive electronic hearing loss diagnosis apparatus including a data storage media and a media player for accessing data on the media. The diagnostic apparatus provides hearing loss diagnostic data to the individual in the form of coded data. A hearing loss professional can use the coded data to further diagnose the hearing loss of the individual.

### CROSS REFERENCE TO RELATED APPLICATION

This application claims the benefit of U.S. Provisional Application No. 60/579,369 filed Jun. 14, 2004, assigned to the assignee of this application and incorporated by reference herein.

### FIELD OF THE INVENTION

The present invention relates to hearing aid testing systems. More particularly, the present invention relates to administering a hearing-aid test on a low-cost, standardized device such as a compact disk (CD) or videotape that is playable on a standard CD/VHS player. The CD/videotape is programmed with a set of tests including frequencies at various amplitudes on different tracks. Each track has verbal instructions that guide the person being tested (the user) to other preprogrammed tracks based upon the user's hearing response to the current track. In so doing, the user ends up in a unique track that then guides him or her to take a related action based upon the test results, for instance, to seek a further test.

### BACKGROUND OF THE INVENTION

More than 25 million Americans have hearing loss, including one out of four people older than 65. Hearing loss may come from infections, strokes, head injuries, some medicines, tumors, other medical problems, or even excessive earwax. It can also result from repeated exposure to very loud noise, such as music, power tools, or jet engines. Changes in the way the ear works as a person ages can also affect hearing.

For most people who have a hearing loss, there are ways to correct or compensate for the problem. If an individual has trouble hearing, that individual can visit a doctor or hearing health care professional to find out if he or she has a hearing loss and, if so, to determine a remedy. The U.S. Food and Drug Administration (FDA) and similar governing bodies in other countries have rules to ensure that treatments for hearing loss—medicines, hearing aids, and other medical devices—are tried and tested.

However, most people do not even know that they have a hearing loss. Typical indications that an individual has hearing loss include: (1) shouting when talking to others, (2) needing the TV or radio turned up louder than other people do, (3) often having to ask people to repeat what they say because the individual cannot quite hear them, especially in groups or when there is background noise, (4) not being able to hear a noise when not facing the direction it is coming from, (5) seeming to hear better out of one ear than the other, (6) having to strain to hear, (7) hearing a persistent hissing or ringing background noise, and (8) not being able to hear a dripping faucet or the high notes of a violin. If an individual experiences one of more of the above indications, the individual should see his or her doctor or hearing health care professional for further testing for potential hearing loss.

To find out what kind of hearing loss the individual has and whether all the parts of the individual's ear are functioning, the person's doctor may want him or her to take a hearing test. A health care professional that specializes in hearing, such as an audiologist, often gives these tests. Audiologists are usually not medical doctors, but they are trained to give hearing tests and interpret the results. Hearing tests are painless.

If the hearing test shows that the individual has a hearing loss, there may be one or more ways to treat it. Possible treatments include medication, surgery, or a hearing aid. Hearing aids can usually help healing loss that involves damage to the inner ear. This type of hearing loss is common in older people as part of the aging process. However, younger people can also have hearing loss from infections or repeated exposure to loud noises.

In a well-known method of testing hearing loss in individuals, the threshold of the individual's hearing is typically measured using a calibrated sound-stimulus-producing device and calibrated headphones. The measurement of the threshold of hearing takes place in an isolated sound room, usually a room where there is very little audible ambient noise. The sound-stimulus-producing device and the calibrated headphones used in the testing are known as an audiometer.

A professional audiologist performs a professional hearing test by using the audiometer to generate pure tones at various frequencies between 125 Hz and 12,000 Hz that are representative of a variety of frequency bands. These tones are transmitted through the headphones of the audiometer to the individual being tested. The intensity or volume of the pure tones is varied until the individual can just barely detect the presence of the tone. For each pure tone, the intensity at which the individual can just barely detect the presence of the tone is known as the individual's air conduction threshold of hearing. Although the threshold of hearing is only one element among several that characterizes an individual's hearing loss, it is the predominant measure traditionally used to acoustically fit a hearing compensation device.

Although the professional test is complete and allows for a thorough diagnostic, most hearing-impaired individuals are not even aware that they are in need of a hearing test, even if some of the aforementioned symptoms exist. What is required is a way to recognize early onset of hearing loss without the need to visit the audiologist.

Indeed, there are some new methods for testing hearing loss, albeit at a less professional level, such as programs available on the Internet. To use such a program, a user logs onto a free hearing test Web site, adjusts his or her computer speaker volume to a supplied test frequency, and uses a mouse to click on various hyperlinks on a Web page on which the user can listen to various tones and determine how many tones he or she is able to hear. The user then is guided to instructional and “next step” pages. There are a number of problems associated with this method. First, most people that have hearing loss are older, and the Internet may truly not be accessible because of their level of use of technology. Second, many low-income families cannot afford computers to run the Internet programs. Lastly, this system does not “pull” users to the site; an individual has to know both that he or she wants to be tested and that a site like this exists (i.e., from advertisements). No business entity could afford to mass market such a site. Therefore, even though some low-cost non-professional hearing tests are available, there exists a need for an improved means for hearing tests that is more accessible, portable and can be driven in the market to reach and test more people.

Another problem with current methods for testing hearing loss is the inability to store user-specific information in a database and provide clear step-by-step guidance on the actions needed to find a solution once a hearing loss problem is detected. In the case of the Internet hearing test Web site previously described, the results of the test are not directed to another step, nor are they available to another entity, i.e., an audiologist. Therefore, an audiologist must retest the same frequencies and re-question the patient. Thus, there exists a need to streamline the testing process so that low-cost non-professional hearing tests lead to a more professional hearing test.

Another problem with both conventional non-professional hearing tests and the audiologist-administered professional hearing test is that the tests are simple frequency versus amplitude tests and do not take into account speech intelligibility issues. For example, even though an individual may have some hearing loss, he or she may be able to function quite normally, whereas others may have limitations in understanding certain spoken words. Thus, there exists a need to address some of these speech intelligibility issues.

Another problem with current testing methods is that the individual being tested has no idea at the hearing test what having a hearing aid would do to improve his or her quality of life. That is, even if the patient in either the non-professional test or the professional test recognizes hearing loss, the patient has no idea what the improvement would be if a corrective hearing aid were used. Thus, the motivation to get the problem fixed is much less than if the individual could experience the benefits of correction at the time of the test.

### SUMMARY OF THE INVENTION

It is therefore an object of this invention to find a way for the mass market of individuals with potential hearing loss to recognize early onset of hearing loss without the need to visit an audiologist.

Another object of this invention is to have an improved means for testing hearing that is more accessible and can be driven in the market to reach and test more people.

Another object of this invention is to streamline the testing process so that low-cost non-professional hearing tests lead to a more professional hearing test.

Another object of this invention is to address speech intelligibility issues at some level in hearing aid tests.

Another object of this invention is to show patients what the result of having a hearing aid would do to improve their quality of life, in order to improve the patients' motivation to fix the problem.

It is another object of the present invention to provide step-by-step guidance on the next steps to be taken once a hearing loss is detected.

The present invention provides a hearing-test stored on a standard low-cost CD or other low-cost devices, such as a videotape or a DVD, that is easily mass marketed as a give-away and is easily used by the mass market. This would allow the mass market of individuals with potential hearing loss to recognize early onset of hearing loss without the need to visit the audiologist. The present invention streamlines the hearing testing process and connects low-cost non-professional hearing tests to a more professional hearing test by providing the results of the CD hearing test to the user as a code that can be quickly identified by a professional, e.g., an audiologist. This invention provides testing of speech intelligibility issues, where the tests are administered around words and based upon the specific results of the hearing test. The present invention also provides step-by-step guidance on the next steps to be taken, once the hearing test stored on the CD detects a hearing loss.

In a preferred embodiment of the present invention, a portable data storage media is provided for use in testing hearing of an individual. The media comprises a plurality of selectably accessible data storage units, e.g., tracks on a CD or DVD. A first of the units includes a hearing test query, which for example causes a frequency tone, word or sentence to be played at a speaker, and instruction data. The instruction data instructs an individual to access a predetermined second of the units in accordance with the results of the hearing test query, for example, based on whether the individual heard the tone or found the word or sentence to be intelligible. The predetermined second unit includes at least one of a hearing test query and instruction data. The instruction data on the second unit instructs the individual to access another predetermined second of the units in accordance with results of the hearing test query of the predetermined second unit, or provides for output of a hearing deficiency code whose value is associated with the predetermined second unit being accessed.

In a preferred embodiment, the instruction data of the predetermined second unit including the provision for the output of the code further instructs the individual to access a predetermined final code unit on the media including the code.

### BRIEF DESCRIPTION OF THE DRAWINGS

Other objects and advantages of the present invention will be apparent from the following detailed description of the presently preferred embodiments, which description should be considered in conjunction with the accompanying drawings in which like references indicate similar elements and in which:

FIG. 1A is a high-level system diagram of a low-cost-hearing testing system that collects user information.

FIG. 1B is a diagram of a low-cost CD on which a hearing testing system is stored.

FIG. 2 is a high-level method of using the low-cost hearing testing system that collects user information.

FIG. 3 shows a flow chart of a method of operating a low-cost hearing testing system.

FIG. 4 is an example of a hearing test checklist.

FIGS. 5A and 5B illustrate a hearing test questionnaire.

### DETAILED DESCRIPTION OF THE INVENTION

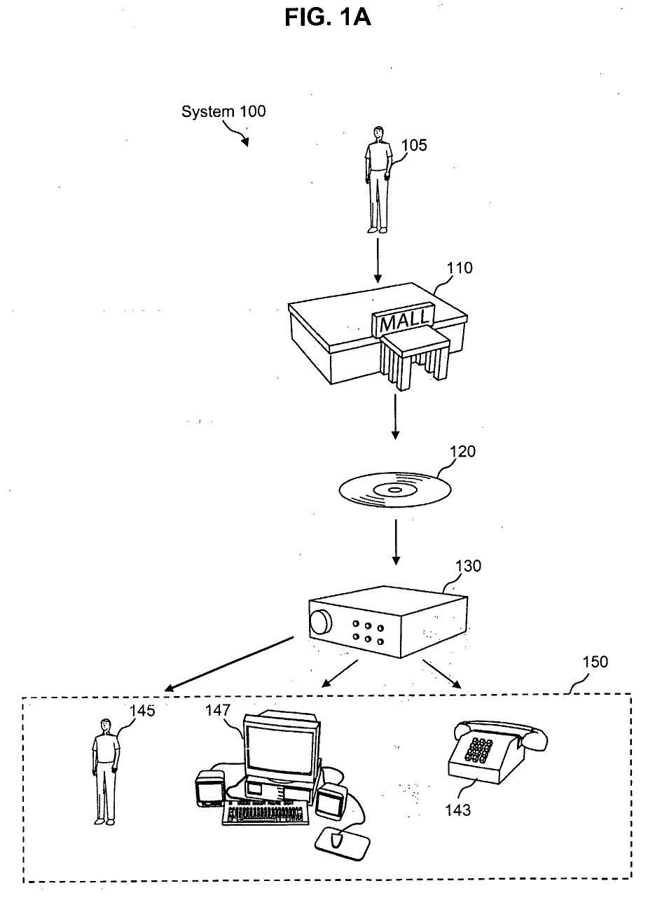

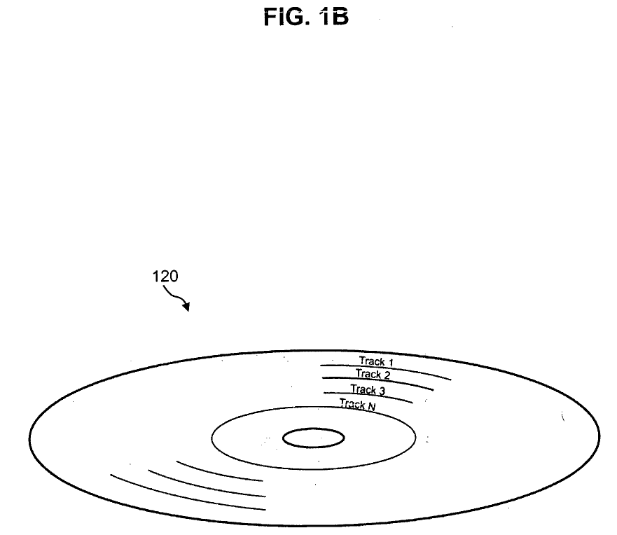

FIG. 1A is a high-level diagram of a preferred system 100 including a user 105, an optimized pick-up location 110, a hearing test CD 120, a CD player 130, a telephone 143, a PC 147, an audiologist 145 and a next hearing test means 150.

User 105 represents the individuals (mass market) on whom a hearing test is to be administered. This is generally any and all individuals, but more specifically, the more than 10% of the population (e.g., 25 million Americans) that have hearing loss, including one out of four people older than 65.

Optimized pick-up location 110 is a location where it would make sense for a hearing test CD 120 to be available toga large number of the general populace. For instance, optimized pick-up location 110 could be in a popular public location, such as a shopping mall where user 105 has easy and frequent access. For example, optimized pick-up location 110 could be a CD music store in a mall, a CD player store, a consumer electronics retail store such as a music showroom, a computer store, a health store, the lobby of a pilot training building, a mail order campaign, the lobby of a nursing home, or a general practitioner's office. Each of these locations is optimized since they have something to do with CD technology, health, mass market testing; older individuals, or selected professionals that may have hearing loss. By providing hearing test CD 120 as a give-away to the mass market focused on where there is need or capability for the hearing test, the large potential market can be tapped and those in need can be assisted in understanding their early onset or advanced hearing loss.

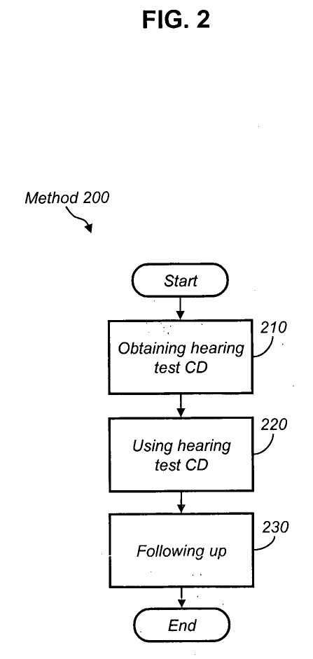

Hearing test CD 120 contains a hearing test that helps determine hearing loss. Hearing test CD 120 also provides follow-up actions for user 105. The hearing test is described in more detail in reference to FIG. 2.

CD player 130 is a standard player that can play hearing test CD 120. Because the hearing test guides the user to select or skip tracks based upon the response of the user 105 to a given track, CD player 130 in system 100 needs to have some means for user 105 to know what track they are on. Almost all conventional CD players come equipped with at least a two-element display to show up to 99 tracks. These 99 tracks available on a standard CD player are adequate for the hearing test to be performed. Note that CD player 130 can be a stand-alone player or part of another electronic device such as a computer or a car music system.

Telephone 143 and PC 147 are conventional communication means enabling user 105 to conduct a second, more thorough, hearing test. After user 105 takes hearing test CD 120 on CD player 130, instructions on hearing test CD 120 guide user 105 to call a toll-free phone number using telephone 143, or to log onto the Internet using PC 147 to connect to a specific Web site, both of which are sources of additional testing. User 105 could also call or visit audiologist 145 directly. Any of these three next steps, defined as a group as next hearing test means 150, can provide user 105 with a second-level hearing test. The second-level hearing test can be performed at the toll-free phone number, the Web site, or audiologist 145.

In a preferred embodiment, hearing test CD 120 provides user 105 with a code to use prior to taking the second-level hearing test that can streamline the testing process. For example, a user 105 that has a severe hearing loss in a given frequency range may be specifically guided to a final test track on hearing test CD 120 that contains a specific code relating to that deficiency. At the second level of testing, user 105 could provide that code and start further testing immediately at the range in which he or she is deficient.

Note that in alternative modes of system 100, hearing test CD 120 can easily be replaced with other devices such as a videotape or DVD. If so, CD player 130 can also be replaced with a standard VHS player for the videotape or DVD player for the DVD.

FIG. 1B illustrates a detailed schematic of hearing test CD 120 with multiple tracks, i.e., Track 1 through Track N. Each track on hearing test CD 120 has information on it organized in such a manner to enable a portion of a hearing test to be performed and instructions to be given to guide user 105 to another track based upon his or her response to the given track.

FIG. 2 illustrates a high-level method 200 of using system 100, including the steps of:

Step 210: Obtaining Hearing Test CD

In this step, user 105 obtains hearing test CD 120 at any of a number of optimized pick-up locations 110. In addition, there can be many other low-cost ways for user 105 to obtain hearing test CD 120. For example, hearing test CD 120 can be sent through a mail-order campaign.

Step 220: Using Hearing Test CD

In this step, user 105 runs hearing test CD 120 on CD player 130 to take the hearing test.

Step 230: Following Up

In this step, based on the follow-up instructions on hearing test CD 120, user 105 is directed (if necessary) to any number of communication means. These means may include using telephone 143 to dial a toll-free phone number provided on hearing test CD 120, using PC 147 to connect to the Internet through a Web site provided on hearing test CD 120, or going directly to audiologist 145 to eventually obtain a second-level hearing test. In this step, user 105 provides the code that is unique to the result of his or her initial hearing test.

For example, a user 105 that has a severe hearing loss in a given frequency range may be specifically guided to a final test track on hearing test CD 120 that contains a specific code relating to that deficiency. At the second level of test, user 105 could provide that code and start further testing immediately at the range in which he or she is deficient. To further prompt user 105 to conduct a second level of test, an added incentive such as cost savings “coupon” can also be provided via means such as in the cover of hearing test CD 120, or web site link to electronic “coupons”.

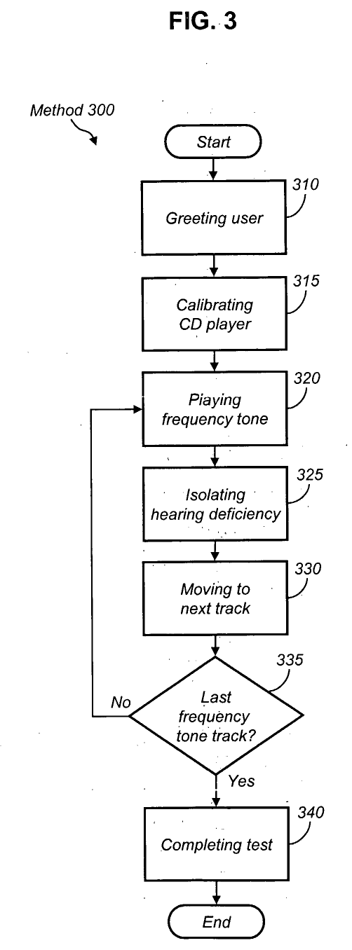

FIG. 3 illustrates a method 300 of conducting a hearing test using hearing test CD 120. The basic concept of method 300 is to guide user 105 from track to track on hearing test CD 120 by verbal commands. Each track has a number of recorded information units, which represent logical steps in the flow of a program. Therefore, the availability of any succeeding tracks is dependent upon the parameters of the current track. In this way, user 105 is guided through a program. The method includes the steps of:

Step 310: Greeting User

In this step, user 105 plays hearing test CD 120 in CD player 130 and is greeted with a message, such as a message to welcome the user, introduce the hearing test, and give general instructions on how to use hearing test CD 120. User 105 is instructed to move to the track number that is used for calibration of CD player 130 to hearing test CD 120. In order to make hearing test CD 120 more “fun” to use, a well-known recording artist such as Robin Williams can record the voice over. This feature will be especially attractive in case of testing young adults and small children. More so, if the costs of hiring using the well-known recording artists are spread across millions of hearing test CD 120's, the overall cost per CD will not increase sharply.

Step 315: Calibrating CD Player

In this step, hearing test CD 120 provides a verbal set of directions that tells user 105 to set the volume on CD player 130 to a level that is “just audible” by a third party with normal hearing, e.g., a person younger than 20 with perfect hearing is asked to set the volume to a minimum level of speech understandability. Setting the volume on CD player 130 to a “just audible” level is an optimal environment to conduct an accurate hearing test, and this volume level is maintained throughout the test. Then user 105 is instructed to play the next group of tracks to find the track by which user 105 can barely hear the tone again. Once user 105 selects this track, the program logic flow, or branch of the logic tree containing tracks calibrated in volume to the selected track, begins. User 105 is then guided to the first frequency test track based upon the correct volume level. Alternatively, hearing test CD 120 can provide a verbal set of directions that tell user 105 to set the volume on CD player 130 to the lowest audible level for user 105. However, this may prevent the determination of absolute loss levels in each frequency range for user 105.

Step 320: Playing Frequency Tone

In this step, user 105 hears a verbal instruction and is played a set frequency tone corresponding to the track on hearing test CD 120. For example, track T3 on hearing test CD 120 plays a frequency tone of 500 Hz.

Step 325: Isolating Hearing Deficiency

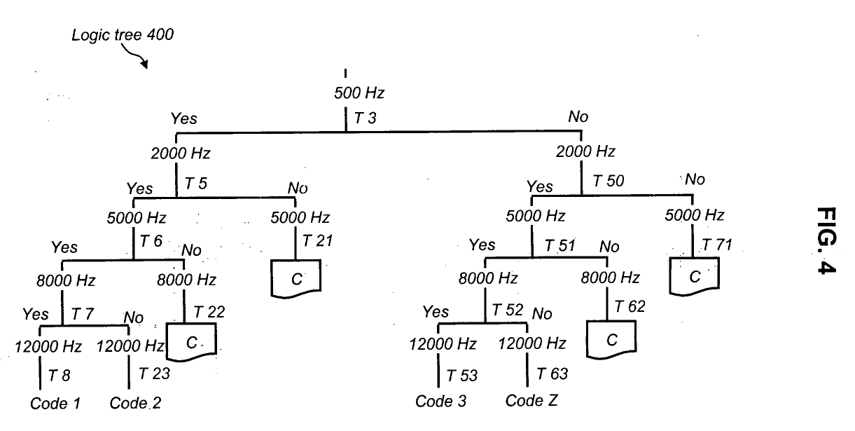

In this step, user 105 is asked to follow a detailed logic tree 400 of tracks on hearing test CD 120. The operation of a sample logic tree 400 is explained in detail in FIG. 4. The specific progression that user 105 takes through logic tree 400 is dependent upon how user 105 responds as to whether each set frequency tone is audible. In this way, hearing test CD 120 determines whether user 105 has a hearing deficiency and, if so, in which specific frequency bands the deficiency occurs.

Step 330: Moving to Next Track

In this step, user 105 is asked to move onto a specific next track of hearing test CD 120 that corresponds both to any hearing deficiencies previously isolated and to the next frequency tone in the series. In the given example, if user 105 has indicated that the 500 Hz tone played in track T3 was audible, track T5 plays a frequency tone of 2000 Hz.

Step 335: Last Frequency Tone Track?

In this decision step, through completion of logic tree 400, hearing test CD 120 determines whether all of the set frequency tones in the hearing test have been played. If so, method 300 proceeds to step 340; if not, method 300 returns to step 320.

Step 340: Completing Test

In this step, user 105 is asked to move on to a specific final track of hearing test CD 120 that congratulates user 105 on completing the hearing test and reports whether the test has found that user 105 may have hearing deficiencies. Tracks corresponding to specific hearing deficiencies assure user 105 that there are many possible means for rectifying the deficiency, and that knowing that one has a deficiency is a first positive step. These tracks also provide user 105 with a code specific to his or her hearing deficiency, which can later be used in conducting a second-level hearing test. User 105 is directed to use next hearing test means 150, i.e., to call a toll-free phone-number or to log onto a specific Web site on the Internet for further testing, or, if the hearing loss is significant, to contact professional audiologist 145 directly. The name and contact information for preferred audiologists can also be provided. Furthermore, an added incentive, such as “coupon” savings on detailed hearing tests or hearing aid accessories, can also be provided to further prompt user 105 to use next hearing test means 150. Method 300 ends. Note that, if the test detected that user 105 does not have any hearing deficiency, then user 105 is congratulated on his/her good hearing and no follow up action may be directed to user 105.

Note that in this mode of operation, user 105 uses a device such as a CD player remote control to advance through tracks on hearing test CD 120, however alternative modes of automated track to track advancement on hearing test CD 120 can easily be suggested.

FIG. 4 shows logic tree 400. Each track, e.g., T3, T5, T7, has a set frequency tone, e.g., 500 Hz, 2000 Hz, 8000 Hz, and instructions articulating decision tree yes or no steps based on user 105's ability to hear the tone. The track at the end of each branch contains a message with a specific code for that branch, e.g., codes 1, 2, 3, and z. Continue branches “C” represent a continuation of the logic flow branch repetition, which has been truncated for the sake of clarity.

In this example, if user 105 provides responses indicating that all tones played were audible, logic tree 400 follows the branch including tracks T3, T5, T6, T7, and T8, and user 105 is assigned a code 1, representing perfect hearing. If user 105 provides responses indicating that all tones played were audible except 500 Hz, logic tree 400 follows the branch including tracks T3, T50, T51, T52, and T53, and user 105 is assigned a code 3 representing this information.

It should be obvious to those skilled in the programming art that there are many combinations of frequency loss; for example, one could hear 500 Hz, 5000 Hz, 8000 Hz, and 12000 Hz, but not 2000 Hz. All these branches lead to unique codes. The number of branches of logic tree 400 is limited only by the available storage, space for the tracks on hearing test CD 120. Depending on the number of possible codes and the number of tracks necessary to arrive at those codes, the branches of logic tree 400 may need to be limited to a certain number of possibilities, or the hearing test may need to span more than one hearing test CD 120.

It should also be obvious to those skilled in the programming art, and basic mathematics, that the results from typical binary logic trees can be K^{N} where N is the number of test frequency nodes and K is the possible outcomes. For example, a basic frequency test that has 5 test frequency nodes, (i.e. 500 Hz, 2000 Hz, 5000 Hz, 8000 Hz, and 12000 Hz) with 2 outcomes (i.e. yes or no), has a logic tree that can be programmed to contain as many as 32 different results.

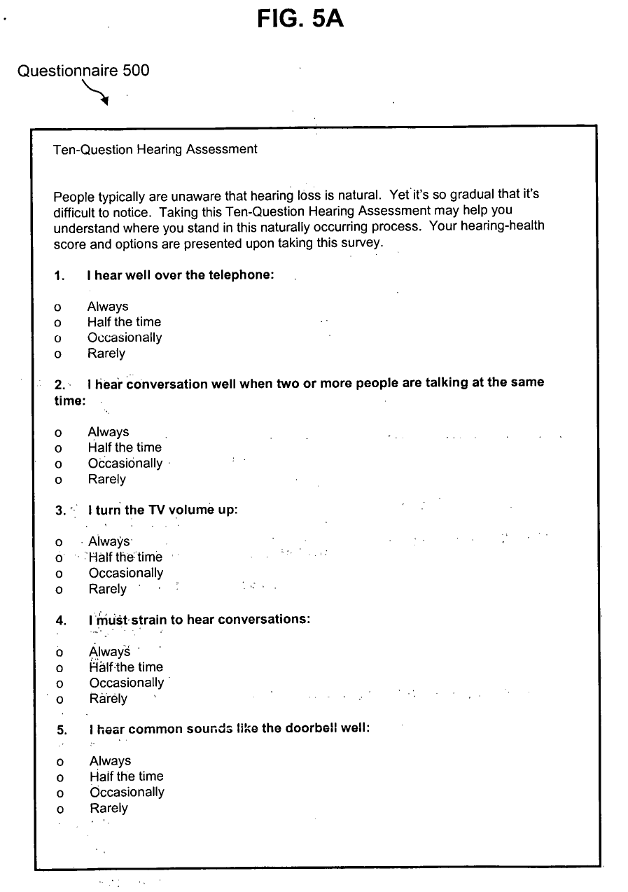

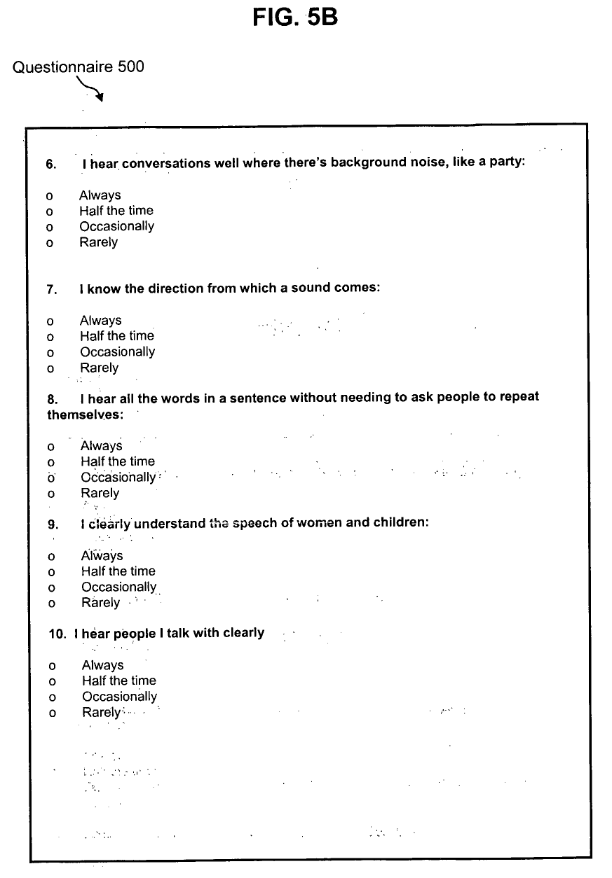

FIGS. 5A and 5B illustrate a questionnaire 500 that can be inserted in the case of hearing test CD 120. By answering questionnaire 500, user 105 can further confirm his or her hearing loss, providing further incentive for user 105 to arrange a second hearing test. These questions, in addition to tones, can be, the basis, of decision branches to obtain even more unique information about user 105.

Thus, the inventive system and method increase the public's awareness that hearing impairment is common, and allows an individual to easily assess any hearing loss and to provide diagnostic results, which are obtained from the assessment, to a hearing professional for use in further assessment of hearing loss.

Although preferred embodiments of the present invention have been described and illustrated, it will be apparent to those skilled in the art that various modifications may be made without departing from the principles of the invention.

What is claimed is:

1. A data storage media for use in testing hearing of an individual, the media comprising:
a plurality of selectably accessible data storage units, wherein a first of the units includes a hearing test query and instruction data, wherein the instruction data instructs an individual to access a predetermined second of the units in accordance with results of the hearing test query,
wherein the predetermined second unit includes at least one of a hearing test query and instruction data, wherein the instruction data on the second unit instructs the individual to access another predetermined second of the units in accordance with results of the hearing test query of the predetermined second unit or provides for output of a final hearing deficiency code whose value is associated with the predetermined second unit being accessed.

2. The media of claim 1, wherein the media is portable.

3. The media of claim 1, wherein the data storage units include at least one of a track on a CD or a DVD.

4. The media of claim 1, wherein the query includes at least one of a frequency tone, word or sentence to be played at a speaker.

5. The media of claim 4, wherein the instruction data instructs, the individual to access the predetermined second of the units if the individual heard the tone or found the word or sentence to be intelligible.

6. The media of claim 1, wherein the instruction data of the predetermined second unit including the provision for the output of the final code further instructs the individual to access a predetermined final code unit on the media, wherein the final code unit includes the final code.

7. A system for diagnosing hearing loss comprising:
a portable media player including a speaker;
a portable data storage media for use in testing hearing of an individual, wherein the media player can access data on the data storage media;
wherein the media includes a plurality of selectably accessible data storage units, wherein a first of the units includes a hearing test query and instruction data, wherein the speaker of the media player generates audible sound based on the hearing test query and, based on the instruction data, instructs an individual to access a predetermined second of the units in accordance with results of the hearing test query,
wherein the predetermined second unit includes at least one of a hearing test query and instruction data, wherein the media player, based on the instruction data on the second unit, instructs the individual to access another predetermined second of the units in accordance with results of the hearing test query of the predetermined second unit or provides for output of a final hearing deficiency code whose value corresponds to the predetermined second unit being accessed.

8. A method of testing hearing of an individual comprising:
providing a portable media player including a speaker; and
providing a portable data storage media for use in testing hearing of an individual using the portable media player, the media comprising:
a plurality of selectably accessible data storage units, wherein a first of the units includes a hearing test query and instruction data, wherein the instruction data instructs an individual to access a predetermined second of the units in accordance with results of the hearing test query,
wherein the predetermined second unit includes at least one of a hearing test query and instruction data, wherein the instruction data on the second unit instructs the individual to access another predetermined second of the units in accordance with results of the hearing test query of the predetermined second unit or provides for output of a final hearing deficiency code whose value corresponds to the predetermined second unit being accessed.

9. The method of claim 8 further comprising:
providing a communications device with the portable media player for communicating the final code to a remote communications device.

10. The method of claim 9, wherein the remote communications device is at least one of a telephone and a modem.

11. The method of claim 8 further comprising:
providing an incentive coupon that the individual can redeem to obtain a discount on additional hearing loss testing where the final code is provided for use in the additional testing.

12. The method of claim 11, wherein the coupon is an electronic coupon.
